(19) United States
(12) Patent Application Publication
(10) Pub. No.: US 2024/0136049 A1
(43) Pub. Date: Apr. 25, 2024

(54) METHOD AND SYSTEM FOR CONVERSATIONAL COGNITIVE STIMULATION

(71) Applicant: Cognifit, Inc, San Francisco, CA (US)

(72) Inventors: Carlos Rodriguez, Madrid (ES); Jon Andoni Duñabeitia, Bilbao (ES)

(21) Appl. No.: 17/973,376

(22) Filed: Oct. 24, 2022

(51) Int. Cl. G16H 20/70; A61B 5/00; A61B 5/16; G10L 15/18; G10L 15/26; G16H 10/20

(52) U.S. Cl. CPC G16H 20/70; A61B 5/165; A61B 5/4803; A61B 5/7257; G10L 15/1815; G10L 15/26; G16H 10/20

(57) ABSTRACT

A method and system are provided for conversational cognitive stimulation performed on an electronic device. The system conducts a lifelike motivational interview, using human speech synthesis, to assess a user's cognitive health, emotional state, topics of interest, sentiment, and overall well-being. This may be accomplished by analyzing vocal and verbal components of the user's responses in the motivational interview through linguistic and paralinguistic analysis. The system may further perform a method to train the user's cognitive abilities according to the results of the motivational interview. The training is customized to each user through customized tasks, questions, and a duration of the training. The system can track changes to the user's cognitive health, abilities, and other areas and make adjustments to the training to promote cognitive stimulation.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to conversational cognitive stimulation, more particularly to a method and system for conversational cognitive stimulation and executing a training program through an electronic device to synthesize human-like speech and imitate lifelike conversational interactions.

### BACKGROUND

Age-related cognitive decline affects many individuals and occurs due to weakened neuron connections that comprise a person's neural network. The severity, and even occurrence, of age-related cognitive decline may be reduced, or even reversed, by strengthening and expanding the person's neural network through cognitive stimulation. As cognitive skills, particularly conceptual cognitive abilities, such as orientation, memory, and language, form the basis for many interactions, including facial recognition and situational adaptability, cognitive decline impacts every facet of an individual's life. Despite an individual's best intentions, many lack sufficient challenges, either in difficulty or frequency, to create a long-term change in conceptual cognition.

Natural interfaces, such as speech, are well-established as highly effective in the production of hormones and neurotransmitters that stimulate the person's neural network. While some professionals, such as certain physicians and therapists, may be trained to reduce conceptual cognitive decline in their patients through natural interfaces, they are notoriously expensive and difficult to access. Thus, many cannot afford such services, and those that can often lack sufficient, consistent time with or attention from such professionals to reduce cognitive decline. However, even those who can access such professionals may fail to receive the full benefit of the conceptual cognitive stimulation. Interactions comprising trust have been shown as more effective to create lasting changes in the production of hormones and neurotransmitters operative to reduce conceptual cognitive decline. However, subjective perceptions and preconceived opinions on behalf of the professional or patient may taint the interaction, creating a lack of trust between the professional and patient, reducing the efficacy of the interaction.

One proposal to prevent cognitive decline, U.S. Publication No. 2015/0,279,226 to Harrison, teaches the use of online video games to test and train an individual's cognitive skills. However, Harrison's games fail to adequately stimulate conceptual cognition and utilize unnatural interfaces, requiring skill and knowledge related to the unnatural interface before cognition may even be stimulated. Some have proposed the use of machines for natural interface communications, such as U.S. Pat. No. 11,315,570 to Alihai. However, these proposals involve passive, one-sided communication wherein the interface offers no feedback to the user. Thus, while the Alihai and other proposals may be operative to receive natural interactions from the use, they do not teach nor suggest interfaces capable of cognitive stimulation.

One proposal, U.S. Pat. No. 10,628,741 to Kaliouby, teaches the use of audio and video interfaces, operating as natural interfaces, operative to interpret a user's emotional cues. While interpreting emotional cues may be beneficial in gaining an individual's trust, Kaliouby remains a passive device operative only to collect and update an individual's emotional cues extracted from the audio and video interfaces. Another, in U.S. Pat. No. 7,764,311 to Bill, has proposed the use of emotional cues for the selection of pre-existing musical recordings in a mood-based music playlisting system. However, Bill does not suggest using the emotional cues for cognitive stimulation. Further, Bill requires cameras to capture images of the individual. The use of cameras, however, may pose an unwelcome invasion of the user's privacy, and therefore could reduce overall trust in the system. Thus, Bill is not only deficient as it is limited to feedback from pre-existing musical recordings, but also fails to adequately establish trust with the user of its system.

Thus, although various proposals have been made to target areas of cognition, such as emotions and abilities, using an assortment of interfaces, none of those in existence combine the characteristics of the present invention. Therefore, there remains a need for a natural interface operative to stimulate an individual's conceptual cognition.

### SUMMARY

The present disclosure is directed to a method and system, implemented on an electronic device, for conversational cognitive stimulation operative to stimulate conceptual cognitive abilities through natural interfaces. In one exemplary embodiment, the system for conversational cognitive stimulation may be configured to enable lifelike conversations between an individual and electronic device which itself is configured to perform speech synthesis through machine learning. It is contemplated that providing a method and system for conversational cognitive stimulation via lifelike conversations according to the disclosure and claims provided below may beneficially engage users in cognitive training.

In accordance with one embodiment, the system may further comprise a personalized training may be conducted to stimulate cognitive skills. The cognitive skills may, for example, be conceptual cognitive skills such as memory, perception, attention, reasoning, coordination, and language, however, a person of ordinary skill will appreciate that the personalized training may stimulate any cognitive skill and the aforementioned are for example only.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the system may comprise an electronic device, such as a tablet, laptop or desktop computer, smartphone, or even a smart speaker. In some embodiments, the electronic device may be any device comprising at least one speaker and at least one microphone. It is contemplated, however, that any speaker or microphone configured to electronically communicate with the electronic device by way of a universal serial bus or other connector or some wireless means such as Bluetooth or Wi-Fi may also satisfy the invention. In some embodiments, the electronic device may further comprise a graphical user interface, a touch-user interface, or any other user-interface as needed or desired.

In order to identify a user, the system may comprise a user profile. The user profile may comprise identifying information, such as, but not limited to, the user's name, date of birth, billing information, and contact information, such as an email address or even a physical address, received by the electronic device. In one embodiment, wherein the electronic device comprises the graphical user interface, the identifying information may be received on the graphical user interface. However, in another embodiment, the identifying information may be received through the at least one microphone. A person of ordinary skill will appreciate that any identifying information as needed or desired may be used and may be received by the electronic device by any suitable means. In addition, the system may be operative to assign the user profile to the identifying information received.

In some embodiments, the system may be operative to identify the user through voice biometrics. In such an embodiment, the identifying information received by the electronic device may be a voice sample. In yet another embodiment, the voice biometrics may comprise a keyword/phrase operative to identify the user. A person of ordinary skill will appreciate all forms of voice biometrics that may be used to identify the user.

In one embodiment, the assessment profile may further comprise any of an emotional profile, a cognitive profile, an interest profile, a sentiment orientation, and a well-being phenotype. Of course, the assessment profile may comprise additional information as needed or desired.

The system may be operative to conduct a motivational interview comprising at least one question operative to elicit information operative to identify the user's thoughts and/or feelings about at least one topic. Indeed, a person of ordinary skill in the art will recognize that motivational interviews are well known evaluation tools that utilize a semi-structured interview process to elicit and explore the user's own motivations for change while providing an atmosphere of acceptance and compassion. As each user's motivations are unique, the system may be configured to tailor the motivational interview to the user. It is contemplated that the system may utilize machine learning and/or artificial intelligence to identify and extract the user's motivation and customize the motivational interview to the user.

Further, the system may be operative to configure the motivational interview to resemble lifelike supportive counseling. It is contemplated that by resembling lifelike supportive counseling the motivational interview may facilitate the atmosphere of acceptance and compassion. For example, the motivational interview may be configured to express empathy to the user. In a further example, the motivational interview may be configured to provide affirmation to the user configured to support the user's self-efficacy.

It is contemplated that the at least one question may be presented in any manner. In one exemplary embodiment, the at least one question may be emitted by the electronic device through the at least one speaker. The system may be operative to synthesize any prompt present through the at least one speaker to imitate human speech. It is contemplated that speech synthesis may provide a more lifelike experience than traditional electronic speech programs, as speech synthesis utilizes machine learning to improve the motivational interview by emulating lifelike interactions. However, the at least one question may be presented in any way as needed or desired, including, without limitation, through any of the previously discussed user interfaces.

In one embodiment, the at least one question may comprise a general question operative to initiate the motivational interview. In some embodiments, the general question may be related to a user's perceived cognitive and/or psychological health. In another embodiment, the at least one question may be a core delight question operative to determine the user's topics of interest. In some embodiments, the topics of interest may be related to a variety of topics a general population may have an interest in. In yet another embodiment, the at least one question may comprise a core arousive question operative to characterize the user's level of psychological activation on at least one controversial topic. In a further embodiment, the at least one question may comprise a follow-up question informed by the response received to another of the at least one question.

The system may be operative to determine the at least one question presented to the user. In some embodiments, the system may customize the at least one question to the user using artificial intelligence. For example, the system may be operative to incorporate the user's name, terminology, and interests into the at least one question. Moreover, the system may determine the type and order of the at least one question in the motivational interview for each user. The system may further determine a number of the at least one question in the motivational interview for each user. It is contemplated that by customizing the motivational interview may better imitate lifelike conversations and may establish a meaningful relationship between the system and the user.

A response to the at least one question in the motivational interview may be received on the electronic device through any means. For example, the response may be a verbal response received at the at least one microphone of the electronic device. However, any form of response to the at least one question capable of being received by the electronic device may be utilized.

In some embodiment, the system may be further operative to present a prompt, such as a verbal affirmation, echo, and verbal agreement, to the user through the electronic device following the response to the at least one question. A person of ordinary skill in the art will appreciate that the prompts and verbal affirmations may be representative of humanlike conversation components. It is contemplated that the prompts and verbal affirmations may be any word and/or phrase and may be configured to encourage the user to provide a further response to the at least one question. Indeed, the system may be operative to customize the word and/or phrase to the user according to the response to the at least one question. A person of ordinary skill in the art will appreciate that the prompts as described are typical components of human conversations, thus providing a more natural and lifelike conversation.

In one embodiment, the response to the at least one question may be analyzed through a linguistic analysis. Linguistic analysis will be understood, by a person of ordinary skill in the art, to be a process operative to interpret a meaning from the response to the at least one question in the motivational interview.

The response to the at least one question may be converted to text. For example, in embodiments where the response to the at least one question is the verbal response, the response may be transcribed. However, it is contemplated that in some embodiments, the response may be a written response and thus may already be in text. It is further contemplated that converting the response to text may occur through any method as needed or desired.

Following converting the response to text, the system may be operative to tokenize the text. The step of tokenizing the text may comprise, as non-limiting examples, text tagging and lemmatization. A person of ordinary skill will appreciate the aforementioned examples are known methods for text processing and that any process for text processing may be utilized.

In some embodiments, the system may be operative to extract an emotional state of the user from the response to the at least one question. The emotional state may be any emotional state known in the art, including, for example, and without limitation pleasure, arousal, and dominance. A person of ordinary skill will recognize that emotional state may be used interchangeably with psychological state or mood and such terminology is incorporated herein by reference.

In one embodiment, a sentiment of the text may be extracted to determine a subjective inflection of the response to the at least one question. The sentiment of the text may, in some embodiments, comprise affective mapping operative to determine connections between aspects of a person's belief system. In another embodiment, the sentiment of the text may comprise identifying an opinion expressed in the response to the at least one question. The opinion may, for example, be a polarity of the response, such as a positive, neutral, or negative opinion on a matter.

In some embodiments, the analysis of the response to the at least one question may be a paralinguistic analysis operative to extract vocal signals from the response to the at least one question. A person of ordinary skill in the art will recognize that vocal signal may relate to any of cognitive dysfunction, emotional state, and meanings of words and/or phrases, among others.

In some embodiments, where the response is the verbal response, the paralinguistic analysis may comprise converting the verbal response to a waveform. A person of ordinary skill in the art will recognize that converting sounds, such as sounds made by the verbal response, to waveforms is well-known in the art and all available methods may be utilized.

The waveform may comprise an amplitude dimension, a frequency dimension, and a time dimension. Each of these dimensions are well-known by a person of ordinary skill in the art and are provided as non-limiting examples.

In one embodiment, the waveform may be analyzed. More particularly, any of the amplitude dimension, frequency dimension, and time dimension may be analyzed. In some embodiments, analyzing the time dimensions may comprise analyzing any of a duration of utterances, articulation rate, and a number and duration of pauses. In another embodiment, analyzing the amplitude dimension may analyze any of an intensity and loudness of the response to the at least one question. In yet a further embodiment, analyzing the frequency dimension may comprise analyzing any of a pitch or fundamental frequency of the response to the at least one question.

In some embodiments, analyzing the waveform may further comprise determining a variability of the waveform. It is contemplated that variability may be related to any of the aforementioned dimensions of the waveform. The variability may be operative to determine changes in cognitive skill levels. In one embodiment, the system may utilize the variability to inform any of the personalized training.

In one embodiment, the waveform may be transformed into a frequency-domain signal comprising a power spectral density and at least one frequency band. A person of ordinary skill in the art will appreciate the use of frequency-domain signals and their relation to paralinguistic analysis. As a non-limiting example, the frequency-domain signals may be utilized to extract the emotional state of the user through a tone, inflection, or any other data represented on the frequency-domain signal. In another non-limiting example, the frequency-domain signals may be utilized to identify cognitive skills, including cognitive dysfunction.

In response to conducting the analysis on the response to the at least one question of the motivational interview, the system may update the assessment profile. It is contemplated that updating the assessment profile may be operative to track changes over multiple motivational interviews.

In one embodiment, the emotional profile may be updated to comprise any of the emotional states identified. In some embodiments, the emotional profile may comprise pre-selected emotions that may be updated. The pre-selected emotions may be basic emotions, core affects, or any other grouping of emotions as needed or desired. It is contemplated that by limiting the emotional profile to pre-selected emotions may be operative to consistently track the emotional state over time.

In another embodiment, the sentiment orientation may be updated to comprise the sentiment of the text.

In a further embodiment, the interest profile may comprise at least one topic of interest specific to the user. The topic of interest may be determined by the analysis of the response to the at least one question in the motivational interview. In some embodiments, the at least one topic of interest may be related to the core-arousive and core-delight questions in the motivational interview.

In yet another embodiment, the cognitive profile may comprise a skill level of at least one cognitive ability.

In one embodiment, the well-being phenotype may comprise any of a user's physical well-being, cognitive well-being, psychological well-being, and social well-being. It is contemplated that well-being phenotype may, for example, be informed by any of the emotional profile, sentiment orientation, interest profile, and cognitive profile.

In some embodiments, the system may perform the personalized training by performing a method of selecting from the user's interest profile one of the at least one topic of interest, generating a training task, presenting the training task to the user, receiving a response to the training task, and if the response is accurate updating the user's profile. It is contemplated that the system may repeat the method until a termination event occurs.

It is contemplated that by generating training tasks related to the user's interest profile, training may be personalized to the user, and the user's engagement with the system may be increased. Increasing the engagement with the system may, in some instances, be operative to increase the efficacy of the training.

The training task may, in some embodiments, comprise a training prompt operative to facilitate an answer from the user. However, in another embodiment, the training prompt may be a sound, such as a beep or a melody. In a further embodiment, the training task may comprise an instruction operative to explain the training to the user.

The system may receive a response to the training task. The response to the training task may be any response requested by the system. For example, the system may request an affirmative response, such as an answer to the question, or even a lack of an affirmative response, such as silence. In some embodiments, a correct response may be associated with the training task.

In some embodiments, the system may be operative to determine if the response to the training task is the correct response. In one embodiment, the system may classify the response to the training task as correct when it is identical to the correct response. However, in another embodiment, the response may be classified as correct even if it is not identical to the correct answer. In such an embodiment, it is contemplated that the response to the training task may be considered partially correct.

If the response to the training task is not classified as correct, the system may, in some embodiments, cue the user to provide a new response to the same training task. In some embodiments, the cue may comprise repeating at least some of the training task. For example, the system may repeat the same training prompt and/or question. However, in another embodiment, the cue may be any cue configured to facilitate a response from the user. The system may receive the new response and may determine if the new response is correct. In some instances, the system may be operative to continue regardless of the accuracy of the new response. However, in some instances, the system may not continue with the personalized training until the correct response is received. It is further contemplated that the system may receive a request to continue and no response to the training task may be received.

However, in another embodiment, the system may be operative to continue with personalized training regardless of the response received.

Following the step of receiving the response, the response to the training task may be analyzed to new data related to the assessment profile. For example, the new data may be a change in the user's cognitive abilities or a change in the user's emotional state. It is contemplated that the new data may be extracted in any manner known in the art. Thus, in some embodiments, the new data may be extracted in the same manner as the information from the motivational interview.

In one embodiment, the new data extracted may be saved to the user's profile. However, in another embodiment, any information, whether new or otherwise, extracted from the personalized training may be saved to the user's profile.

In one embodiment, the system may be operative to repeat the personalized training until a termination event occurs. The termination event may, for example, and without limitation, be any of a negative emotional state, a request to terminate, or a number of incorrect responses.

In some embodiments, the motivational interview and the personalized training may be conducted consecutively. However, in another embodiment, the motivational interview and the personalized training may be conducted independently. In yet another embodiment, the motivational interview and personalized training may be conducted simultaneously.

Several advantages of one or more aspects of the method and system for conversational cognitive stimulation are that they:
a.) Utilize natural interfaces to permit access to cognitive stimulation to all skill levels;
b.) Increase accessibility to cognitive training programs;
c.) Provide personalized training of cognitive skills;
d.) Create a long-term change in the user's body chemistry;
e.) Simulate on electronic devices life-like conversations through personalized interactions and reflective listening; and
f.) Affirm success and achievements of the user.

Thus, it is an object of this method and system to provide access to conceptual cognitive stimulation through a personalized training.

It is a further object of this method and system to analyze a user's interest to assign personalized training in a manner configured to increase user engagement. It is yet another object of this method and system to reduce frustration with personalized training by analyzing a user's overall well-being. It is still a further object of this invention to help the user establish a cognitive training routine.

In yet another object of this method and system to provide a highly engaging experience and life-like conversation interactions conducted on electronic devices.

It is a further object of this method and system to utilize the natural interface of speech to exchange conceptual information.

It is an object of this system and method to boost the production of hormones and neurotransmitters to reinforce neural networks.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 illustrates an exemplary embodiment of the system for conversational cognitive stimulation

FIG. 2 shows an exemplary embodiment of a computing device shown in FIG. 1.

FIG. 3 is a flowchart depicting an exemplary embodiment of a method for one aspect of the system for conversational cognitive stimulation as carried out in the exemplary motivational interview module shown in FIG. 2.

FIG. 4 shows an exemplary embodiment of a linguistic analysis that may be carried out in the exemplary method shown in FIG. 3.

FIG. 5 is a flowchart depicting an exemplary embodiment of a paralinguistic analysis that may be carried out in the exemplary method shown in FIG. 3.

FIG. 6 is a flowchart depicting an exemplary embodiment of a method for personalized training.

FIG. 7 provides an exemplary embodiment of a feedback loop to assess and train cognitive abilities.

The disclosed embodiments may be better understood by referring to the figures in the attached drawings, as provided below. The attached figures are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

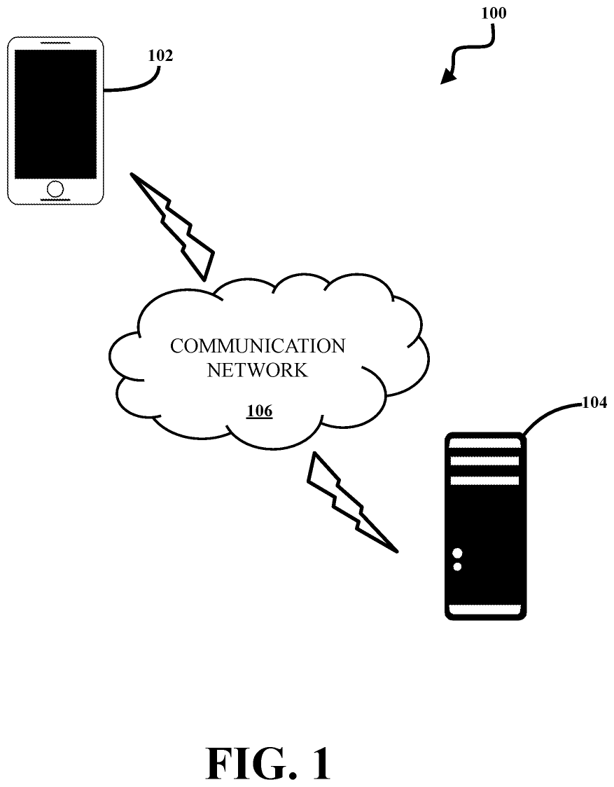

A description of an embodiment of a method and system for customizable cognitive rehabilitation is now described followed by a discussion of the operation of various components within the system. In this regard, FIG. 1 illustrates an exemplary embodiment of the system for conversational cognitive stimulation 100 which includes at least one electronic device 102 and 104. FIG. 1 illustrates two electronic devices 102 and 104 coupled via a communication network 106. Each of the electronic devices may be embodied as a personal computer, tablet computer, laptop computer, smartphone, or smart speaker. In some embodiments, the electronic devices may comprise at least one speaker and at least one microphone. However, in another embodiment, the electronic device may be able couple with at least one speaker and at least one microphone, for example, and without limitation headphones. Thus, any electronic device that may use or connect with at least one speaker and at least one microphone. Notably, the communication network can use one or more various communication types such as, for example, and without limitation, cellular and Wi-Fi communications.

A user of the at least one electronic devices 102 and 104 may use their devices to access the system 100 for conversational cognitive stimulation. In one embodiment, the user may access the system 100 on a graphical user interface on at least one electronic device 102. However, in another embodiment, the user may access the system 100 through the at least one speaker. Thus, the system 100 may be accessed through a verbal prompt.

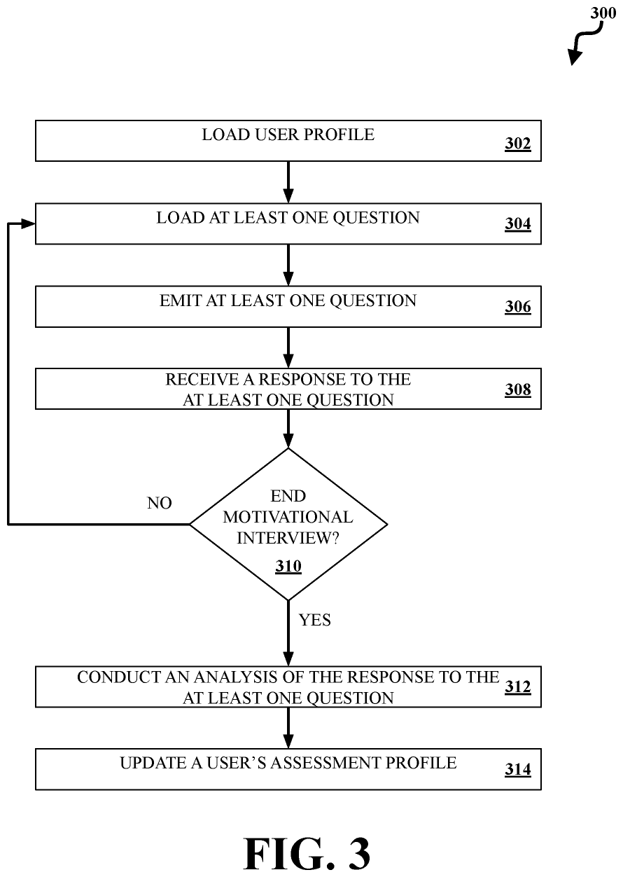

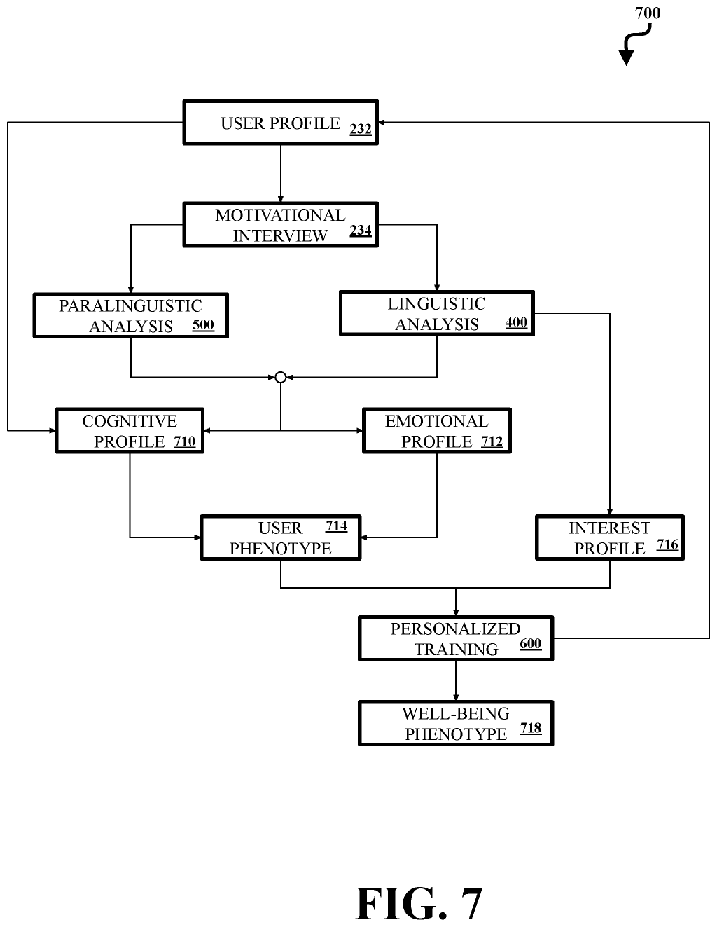

It is contemplated that a user profile may be associated with the user. The user profile may be operative to associate information related to conversational cognitive stimulation with the user. Thus, information extracted during the methods described in FIGS. 3-6 may be associated with the user profile. One exemplary embodiment of this association is a feedback loop shown in FIG. 7. The user profile may further comprise a user's identifying information, including, as non-limiting examples, the user's name, date of birth, billing information, and contact information, such as an email address or even a physical address. The user's identifying information may be received by the at least one electronic device 102 and 104. In some embodiments, the user's identifying information may be received by the at least one electronic device 102 and 104 on the graphical user interface. However, the user's identifying information may be received by the at least one speaker associated with the at least one electronic device 102 and 104 in one embodiment. In yet another embodiment, the user's identifying information may be received by a combination of the aforementioned embodiments or in any other means for receiving that may be contemplated.

The at least one electronic device 102 and 104 may access the user account in any method known in the art. For example, the user account may be accessed by the graphical interface. However, in another embodiment, the system may be operative to access the user account by receiving a verbal cue. The verbal cue may, for example, and without limitation, comprise a keyword/phrase operative to identify the user. For example, the keyword/phrase may be a username associated with the account. In one embodiment, the system may use voice biometrics operative to identify the user through a voice sample.

In some embodiments, one of the at least one electronic device 104 may be a server. The user profile may be stored on the server and may be accessed from any electronic device 102. Thus, the server may be operative to permit access to the user may be able to access their account on a plurality of electronic devices.

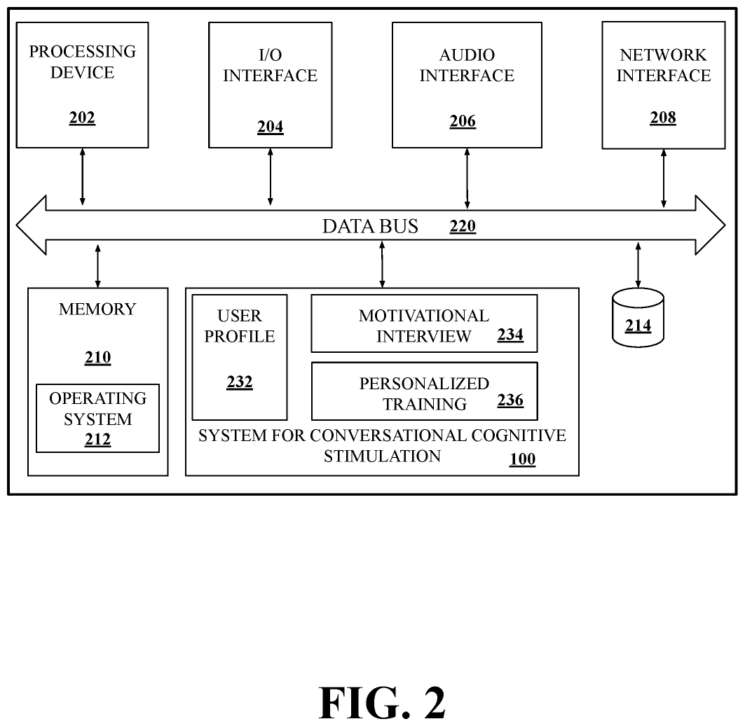

FIG. 2 illustrates an exemplary embodiment of the at least one electronic devices 102 and 104 shown in FIG. 1. As described earlier, electronic device 102 may be a personal computer, tablet computer, laptop computer, smartphone, or smart speaker, but may also be embodied in any one of a wide variety of wired and/or wireless computing devices. As shown in FIG. 2 electronic device 102 includes a processing device (processor) 202, an input/output interface 204, an audio interface 206, a network interface 208, a memory 210, an operating system 412, and a mass storage 214 each communicated across a local data bus system 220. Additionally, electronic device 102 incorporates a system for conversational cognitive stimulation 100, which is depicted as including a user profile 232, a motivational interview 234, and a personalized training 236, although the location of information 232, 234, and 236 could vary.

The processing device 202 may include any custom made or commercially available processor, a central processing unit (CPU) or an auxiliary processor among several processors associated with the electronic device 102, a semiconductor based microprocessor (in the form of a microchip), a microprocessor, one or more application specific integrated circuits (ASCICs), a plurality of suitably configured digital logic gates, and other electrical configurations comprising discrete elements both individually and in various combinations to coordinate the overall operation of the system.

The memory 210 can include any one of a combination of volatile memory elements (e.g., random-access memory (RAM, such as DRAM, and SRAM, etc.)) and nonvolatile memory elements. The memory typically comprises native operating system 212, one or more native applications, emulation systems, or emulated applications for any of a variety of operating systems and/or emulated hardware platforms, emulated operating systems, etc. For example, the applications may include application specific software which may comprise some or all the components of the electronic device 102. In accordance with such embodiments, the components are stored In memory and executed by the processing device. Note that although illustrated separately in FIG. 2, the system and method for conversational stimulation 100 may be resident in memory such as memory 210.

Audio interface 206 is any means operative to receive audio signals and may permit the users to access the system for conversational cognitive stimulation 100. In some embodiments, the audio interface 206 may be fully integrated in the electronic device 102. However, in another embodiment, the audio interface 206 may be removably connected to the electronic device 102, for example as a pair of headphones. It is contemplated the audio interface may comprise at least one speaker and at least one microphone. The at least one speaker may be operative to present a variety of stimuli, discussed in more reference with FIGS. 3-7. The at least one microphone may be operative to receive audio input, such as speech. The at least one microphone and the at least one speaker are widely recognized and any embodiment as needed or desired may be utilized.

In some embodiments, the electronic device may further comprise a graphical display or touchscreen interface, not pictured. Touchscreen interface may be configured to detect contact within the display area of the display and provide some functionality such as on-screen buttons, menus, keyboards, etc. that allow users to navigate user interfaces by touch.

One of ordinary skill in the art will appreciate that the memory 210 can, and typically will, comprise other components which have been omitted for purposes of brevity. Note that in the context of this disclosure, a non-transitory computer-readable medium stores one or more programs for use by or in connection with an instruction execution system, apparatus, or device. With further reference to FIG. 2, the network interface device 208 comprises various components used to transmit and/or receive data over a networked environment such as depicted in FIG. 1, When such components are embodied as an application, the one or more components may be stored on a non-transitory computer readable medium and executed by the processing device.

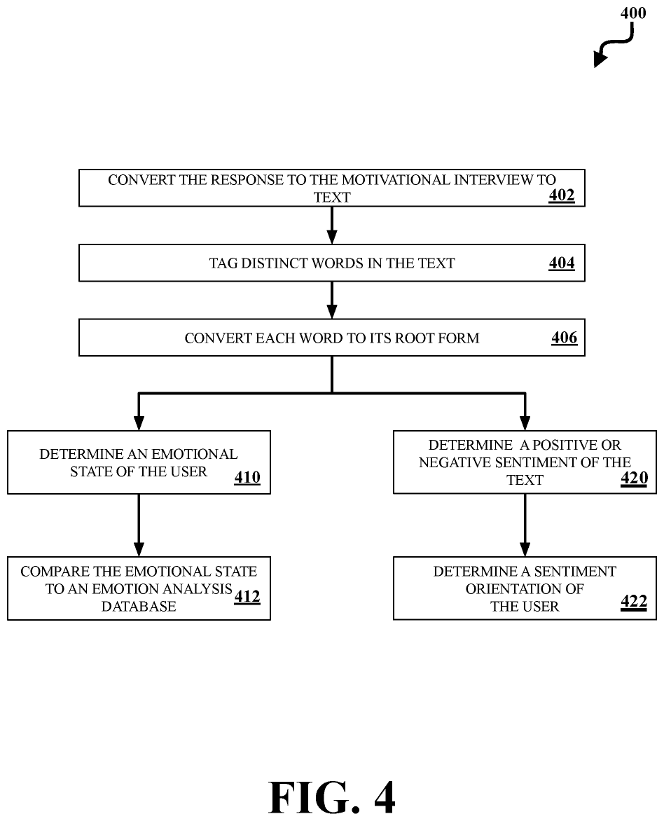

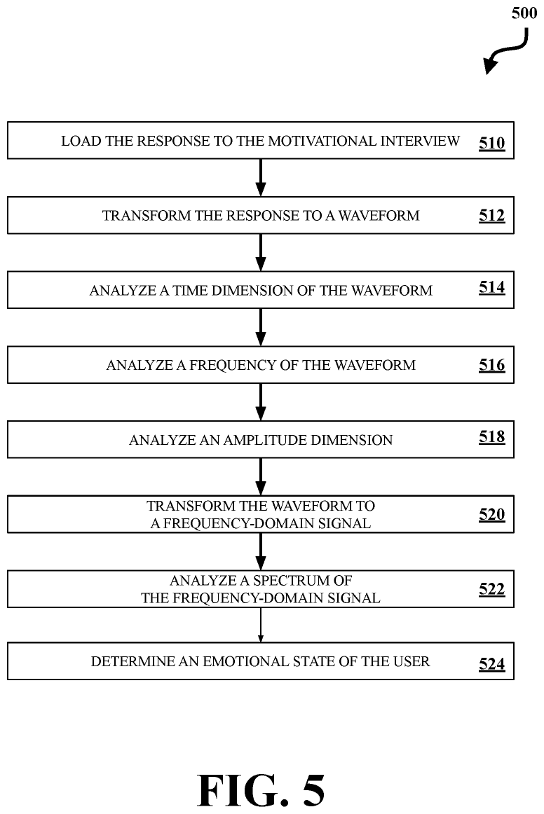

FIG. 3 comprises a chart depicting a method that enables one aspect of the system for conversational cognitive stimulation as carried out in the exemplary motivational interview module shown in FIG. 2. More particularly, and returning to FIG. 3, the system at such module may be configured to load the user profile 302, load at least one question 304, and emit at least one question 306 to the user. A response to the at least one question 306 may be received and an analysis 312 may be conducted on the response. In some embodiments, the analysis may comprise a linguistic analysis and a paralinguistic analysis. These analyses will be discussed in further detail below with reference to FIGS. 4 and 5. Returning to FIG. 3, the user's profile may be updated 314 to reflect any information extracted during the analysis of the response. For example, the user profile may be updated according to the feedback loop shown in FIG. 7. Of course, the user's profile may be updated 314 according to

The user profile may, in some embodiments, be associated with the electronic device. Accessing the system for conversational cognitive stimulation on the electronic device may automatically load the user profile. However, in another embodiment each time the user accesses the system the user's identifying information may be received to load the user profile.

Referring again to FIG. 3, the user profile loaded in step 302 may comprise the user's identifying information, as discussed with reference to FIG. 1, and an assessment profile. The assessment profile may comprise any of an emotional profile, a cognitive profile, an interest profile comprising at least one topic of interest, a sentiment orientation, and a well-being phenotype. Each of these profiles will be discussed in more detail below. A person of ordinary skill will recognize that the aforementioned assessment profiles are provided for example only and any profile as needed or desired may be utilized.

The steps of loading at least one question 304, emitting at least one question 306, and receiving the response 308 to the at least one question may define a motivational interview. It is contemplated that the motivational interview may repeat steps 304, 306, and 308 as many times as needed or desired. Thus, in some embodiments, the motivational interview may only comprise one iteration of steps 304, 306, and 308. However, the motivational interview may comprise two, three, four, five, or even more iterations of steps 304, 306, and 308. It is contemplated that the motivational interview, steps 304, 306, and 308, may end upon the occurrence of an event.

In some embodiments, the at least one question of the motivational interview may be a series of predetermined questions and the event may be receiving a response to each of the series of pre-determined questions. However, in another embodiment, the motivational interview may be customized to the user. Thus, a number of the at least one question and their contents may be specific to the user. In a further embodiment, the motivational interview may comprise the series of pre-determined questions and at least one customized question. In such an embodiment, it is contemplated that the system may be operative to determine the end of the motivational interview. In other embodiments, the event that ends the motivational interview may be a passage of time, a received request, a collection of sufficient data, or any other event as needed or desired.

The at least one question may be any questions operative to determine a user's thoughts and feelings. In some embodiments, the at least one question may be an open-ended question. However, in another embodiment, the at least one question may be a closed-ended question. Any of the at least one questions may be customized to the user. For example, the system may customize the at least one question with information stored in the user's assessment profile, such as their name, topics of interest, responses to another of the at least one question, or even age or geographic specific questions.

In some embodiments, the at least one question may comprise a grand tour question. The grand tour question may be a general question operative to initiate the motivational interview. The general question may, in one embodiment, be related to a user's self-image. For example, the general questions may be related to any of the user's self-perceived cognitive health, psychological health, physical health, and social health. As non-limiting examples, the grand tour question may be broad questions such as “How are you?” or “What have you been up to?” or tailored questions such as “How was your doctor's appointment today?” It is contemplated that the system may utilize machine learning to customize the grand tour questions to each user. A person of ordinary skill in the art will recognize that the grand tour questions may be related to a variety of topics and the aforementioned are provided as non-limiting examples only.

In another embodiment, the at least one question may comprise a core delight question. The core delight question may target at least one topic of interest to determine the user's topics of interest. It is contemplated that the topics of interest may be any topic that an individual may have an interest in. In some embodiments, the topic may be based on general topics in the area of entertainment. For example, the general topics may be related to any of music and dance, games and sports, literature, theater and cinema, animals, travel, and food. Of course, the topics of interest may be any topic of interest of the user. In some embodiments, the system may be operative to further narrow the topics of interest to specific topics.

In yet another embodiment, the at least one question may be a core arousive question operative to characterize a level of psychological activation. More particularly, the core arousive question may characterize the level of psychological activation on at least one controversial topic. For example, the at least one controversial topic may be related to any of politics, people, money, and sex. A person of ordinary skill will recognize that the provided at least one controversial topic are provided for example only and any topic that may be considered controversial may be utilized. As individuals vary on what they may consider controversial, the system may be configured to determine what topics the user considers controversial. The system may, in some embodiments, be operative to determine related controversial topics.

In a further embodiment, the at least one question may be a follow-up question. The follow-up question may be any question, including, in some embodiments, questions determined by the user's response to any of the at least one question. In one embodiment, the follow-up question may be operative to request further details relating to any of the at least one question. In such an embodiment, it is contemplated that the system may be operative to determine instances where the follow-up question may be beneficial. For example, the system may be operative to tailor the follow-up question to the response to the at least one question to expand on the information extracted. Such as, and without limitation to, “Can you tell me more?” and “How did that make you feel?” In some embodiments, the system may be operative to generate the follow-up question specific to the user based on previous motivational interviews and/or the user profile.

It is contemplated that the motivational interview may comprise a combination of the aforementioned embodiments of the at least one question. Thus, the at least one question presented during the motivational interview may be selected from any of the grand tour questions, core delight questions, core arousive questions, and follow-up questions. Presenting a range of questions relating to a variety of areas is contemplated to extract responses related to a wider range of topics from the user and thus extract more information relating to the user.

In some embodiment, not shown, the system may be operative to emit a prompt in response to the at least one question. The prompt may be operative to encourage the user to provide a further response to the at least one question. For example, phrases such as “tell me more,” or even sounds like “hmm” may be utilized. In some embodiments, the prompt may be operative to expand on the responses received to the at least one question, such as and without limitation to “tell me more.” For example, the prompt may be determined by the response received to the at least one question. In one embodiment the system may be operative to determine whether more data is required from the question and/or response. In such an embodiment, the prompt may be configured to build upon the at least one question. In another embodiment, the prompt may be configured to expand or narrow the at least one question. Certain exemplary examples include where the prompt requests clarification, asks the user to repeat the response to the at least one question, or any other prompt that a person of ordinary skill in the art may recognize as beneficial. Of course, each prompt may be dependent on the at least one question and the response to the at least one question. As such, the system may determine a relevant prompt.

While described as being in response to the at least one question, the prompt may be emitted at any point following the response. In some embodiments, the prompt may even be related to the response to the at least one question received in another motivational interview.

In another embodiment, the system may be operative to emit a verbal affirmation, a verbal agreement, an echo, or any other acknowledgment of the user's response to the at least one question. The echo may, in some embodiments, comprise the system repeating and/or summarizing any of the responses to the at least one question. In some embodiments, the system may be operative to extract a critical aspect of the response to the at least one question and the echo may comprise the critical aspect. A person of ordinary skill in the art will recognize that by emitting the echo the system may simulate lifelike human conversations.

The step of conducting an analysis 312 may be any type of analysis operative to extract information from the response to the at least one question. Two exemplary forms of analysis are discussed with reference to FIGS. 4 and 5 below. In some embodiments, as shown in FIG. 3, the analysis may occur following the motivational interview. However, in another embodiment, the analysis may occur during each iteration of the motivational interview. In yet another embodiment, the analysis may occur at multiple points throughout the motivational interview.

Continuing with FIG. 3, the user's assessment profile may be updated 314. The step of updating the user's assessment profile 314 may comprise associating the analysis of the response to the at least one question (step 312) with the user's assessment profile. It is contemplated that the user's assessment profile may comprise the analysis from each motivational interview conducted. For example, if the user has completed one motivational interview only one analysis may be stored in the assessment profile, however, if two motivational interviews have been completed, two analyses may be stored in the assessment profile. In yet another embodiment, the user's assessment profile may comprise a set number of analyses. The set number of analyses may be any number of analyses as needed or desired and may comprise the most recent motivational interviews. In a further embodiment, the analyses may be stored in the assessment profile for a period of time. The step of updating the user's assessment profile 314 is discussed in more detail below.

One exemplary form of analysis is described in FIG. 4. More particularly, FIG. 4 illustrates a linguistic analysis 400 that may be conducted on the motivational interview (FIG. 3 steps 304, 306, and 308). The linguistic analysis 400 may comprise converting the response to the at least one question in the motivational interview to text 402, tagging distinct words 404, and converting each word to its root form 406.

The step of converting the response to text 402 may utilize any form of speech-to-text conversion as needed or desired. In one non-limiting example, converting the response to text 402 may use speech-to-text automated transcription protocols. However, a person of ordinary skill in the art will recognize a wide variety of speech-to-text conversions available in the art that may be used to practice the current disclosure.

A person of ordinary skill will recognize the step of tagging distinct words 404 may utilize any language processing means known in the art. The system may be operative to identify individual words from the text and parse the text into sentences. In some embodiments, the system may be operative to identify a part of speech, such as a noun, verb, and adjective, for any of the individual words. In one embodiment, the tagging distinct words 404 may comprise identifying punctuation marks in the text and removing at least some of the punctuation marks from the text. Thus, the contents of the text itself may be isolated.

Each word may be converted to its root form 406. In one embodiment, each word may be converted to its root form 406 through a process known in the art as lemmatization. The process of lemmatization typically comprises determining a meaning of each word and using the meaning to convert the word to its root form. For example, lemmatization may be operative to determine when the word is in a derivative form and identify an associated derivative. The associated derivative may be the root form of the word. However, in some embodiments, there may be an additional step required to convert the word to its root form. The aforementioned lemmatization process is only one of numerous manners in which lemmatization may be performed. A person of ordinary skill in the art will recognize that lemmatization may vary and any variation, including those not described herein, may be utilized to practice the invention.

In another embodiment, each word may be converted to its root form 406 through a process known as stemming. Stemming comprises removing a tail of each word to extract the root word. The tail may be a set number of letters or syllables removed from each word related to a suffix. In some embodiments, stemming may occur on each word in the response. However, in another embodiment, stemming may occur on less than all the words in the response. For example, stemming may occur on words comprising at least a specified number of letters, at least a certain number of syllables, or were tagged with a specific part of speech in the previous step 404.

The aforementioned embodiments of converting each word to its root form 406 are provided as non-limiting examples and any manner contemplated in the art may be utilized. As further non-limiting examples, a morphological analysis, a semitic root extraction, or even a hybrid analysis.

In some embodiments, the step of tagging distinct words 404 and converting each word to its root form 406 may utilize a natural language processor. For example, the natural language processor may comprise a means for automatically tagging distinct words in the text. The use of natural language processing may, in some embodiments, tokenize the text. Natural language processing may further be operative to represent the text in strings, in which each string is associated with a sentence in the text. A person of ordinary skill may recognize that natural language processing may be particularly advantageous as it is operative to extract a meaning, in real-time, from the response.

Following converting each word to its root form 406, the system may be operative to determine an emotional state of the user 410. A person of ordinary skill will recognize that the use of words and phrases may be indicative of the emotional state of the user. In some embodiments, the emotional state associated with the words and phrases may be unique to each user. Thus, the system may become more accurate with continued use of the system. However, in other embodiments, the emotional state associated with the words and phrases may be universal to all users. In yet a further embodiment, the emotional state of some words and phrases may be unique to the user, while the emotional state of other words and phrases may be universal to all users.

Following the step of determining the emotional state of the user 410, the determined emotional state may be compared to an emotion analysis database (step 412). The emotion analysis database may, in some embodiments, comprise a global database comprising at least one emotion. In another embodiment, the emotion analysis database may comprise a database of the user's emotional states. For example, the database of the user's emotional states may comprise emotional states and emotions determined from previous motivational interviews, training, or other data of the user.

The emotional state of the user may be stored in the assessment profile. More particularly, the emotional state of the user may be stored in the emotional profile.

It is contemplated that the system may be further operative to determine a psychological state of the user. Further, the system may be operative to determine a mood of the user. It is contemplated that the psychological state and mood of the user may be determined by the same process as determining the emotional state of the user 410. However, a person of ordinary skill will recognize other manners of determining the psychological state and mood of the user which may be utilized in the current invention.

In some embodiments, the emotional state of the user may be determined for the motivational interview in its entirety. In another embodiment, the emotional state of the user may be determined for each of the responses to the at least one question in the motivational interview. In yet a further embodiment, the system may be operative to determine the emotional state of the motivational interview in its entirety and individual responses to the at least one question.

In some embodiments, the emotional state of the user may be specific to at least one pre-selected emotion. It is contemplated that limiting the emotional state to pre-selected emotions may be operative to permit comparison of a same emotion over a period of time. Further, it may allow the tracking of the emotional state in a uniform manner. For example, the at least one pre-selected emotion may be one, two, three, four, or even more emotions. Some non-limiting examples of the at least one pre-selected emotions include, fear, anger, admiration, happiness, sadness, interest, excitement, pleasure, nostalgia, arousal, dominance, confusion, or any other emotion that may be recognized. In one embodiment, the at least one pre-selected emotion may be three pre-selected emotions. As one example, the three pre-selected emotions may be associated with core affects, which a person of ordinary skill will recognize as emotions relating to reward, punishment, and stress. In another example, the three pre-selected emotions may be related to emotions such as pleasure, arousal, and dominance. However, these examples are provided as non-limiting examples only and should be construed to limit the invention.

The emotional state of the user may be determined by comparing the at least one pre-selected emotion to another of the at least one pre-selected emotion. In some embodiments, the system may assign each of the at least one pre-selected emotion a weight in the emotional state of the user. In some embodiments, the weight may be determined by a strength of the pre-selected emotion in the motivational interview. However, in other embodiments, the weight may be a predetermined weight, a historic weight, or any other weight that may be needed or desired. Of course, the weight of the emotional state of the user may be determined according to any method as needed or desired and the provided embodiments are provided as non-limiting examples.

In the exemplary embodiment shown in FIG. 4, the system may further analyze a subjective dimension by determining a sentiment of the text 420 and determining the sentiment orientation of the user 422. The sentiment of the text may, in some embodiments, be a polarity, such as positive, negative, or neutral sentiment of the text. In another embodiment, the sentiment may be an attitude, thought, opinion, emotion, judgment, or any other sentiment that may be recognized in the art. The aforementioned examples of sentiment are provided for example only and are not limiting.

In one embodiment, the system may determine a sentiment orientation of the user (step 422) from the sentiment of the text. The sentiment orientation may be the sentiment of any of the text. It is contemplated that the sentiment of the text may be extracted from each of the responses to the at least one question. In one embodiment, the sentiment of the text may be determined (step 420) by the responses received to each of the at least one question. In another embodiment, the sentiment of the text may determine (step 420) a connotation for the words in the text. In yet another embodiment, the sentiment may be determined for any response to the at least one question in the same group of questions. For example, the sentiment may be determined for each of the grand tour questions, core delight questions, core arousive questions, and follow-up questions. In yet another embodiment, the sentiment may be determined for any portion of the text that may be needed or desired.

In some embodiments, further information extracted from the motivational interview may inform the sentiment orientation of the user. For example, any subjective or objective aspect of the motivational interview may be operative to inform the sentiment orientation.

It is contemplated that determining the sentiment orientation of the user may be utilized to track the user's overall well-being. Further, the general sentiment may be operative to track changes in the user's interaction with the system over time to inform treatment. A person of ordinary skill will appreciate that a wide variety of uses for the general sentiment of the user, any of which may be utilized here.

FIG. 5 illustrates a method for paralinguistic analysis 500 that may be conducted on the motivational interview in step 312 of FIG. 3. The paralinguistic analysis 500 may comprise the steps of loading the response to the at least one question in the motivational interview 510, transform the response to a waveform 512, analyze a time dimension 514, analyze a frequency of the waveform 516, analyze an amplitude dimension 518, transform the waveform to a frequency-domain signal 520, analyze a spectrum of the frequency-domain signal 520, and determine an emotional state of the user 524.

In some embodiment, the response to the at least one question in the motivational interview may be a verbal response received by the at least one microphone of the electronic device. A person of ordinary skill in the art will appreciate that sounds, such as verbal responses, are received as electrical currents on microphones. These electrical currents are often represented as a time-based waveform wherein changes in electrical currents over time are represented. In some embodiments, this electrical waveform may be the waveform disclosed in step 512. However, in another embodiment, the electrical waveform may be different from the waveform and additional transformation, as known in the art, may be required.

A person of ordinary skill in the art will appreciate that time waveforms generally comprise a time dimension and an amplitude dimension. The time dimension and amplitude may inform the frequency.

The step of analyzing the time dimension 514 may be operative to analyze any of a duration of utterances, articulation rate, and number and duration of pauses. The duration of utterances may, for example, be a duration of the response received to the motivational interview. More particularly, the duration of utterances may be a duration of each response to the at least one question that comprises the motivational interview. The articulation rate may be a pace at which the response is received. A person of ordinary skill in the art will appreciate that articulation may be used interchangeably with a speaking rate, speech rate, or any other term that may be used to describe the pace of the response.

The step of analyzing the amplitude dimension 518 may be operative to analyze any of an intensity and loudness of the response received to the motivational interview. A person of ordinary skill will appreciate that amplitude may be used synonymously to describe a power of a signal. For example, a greater amplitude may be related to a louder verbal response received and a smaller amplitude be representative of a quieter verbal response. The step of analyzing the amplitude dimension 518 may further comprise analyzing any of a vocal shimmer, jitter, pitch perturbation, vocal entropy, and quality. Each of the above are well-known terms in the art and will be understood by a person of ordinary skill in the art. Furthermore, a person of ordinary skill in the art will appreciate that vocal shimmer and jitter may be used to represent the intensity and loudness, respectively, of the response.

The step of analyzing the frequency 516 may be analyze a pitch of the response received to the motivational interview. In some embodiments, the pitch may be representative of a highness or lowness of the verbal response. Further, the frequency may analyze a fundamental frequency of the response received to the motivational interview. The fundamental frequency is a term in the art and its ordinary meaning is incorporated herein.

In one embodiment, the at least one cognitive skill may be a conceptual cognitive skill. Conceptual cognitive skills may comprise any of attention, perception, memory, reasoning, coordination, and language. More particularly, the conceptual cognitive skills may comprise any of focused attention, divided attention, inhibition, updating, visual perception, estimation, auditory perception, recognition, working memory, short-term memory, long term memory, auditory short-term memory, non-verbal memory, contextual memory, naming, processing speed, planning, shifting, reaction time, production, understanding, and fluency. However, a person of ordinary skill in the art will appreciate that the aforementioned conceptual cognitive skills are provided as, example only and without limitation. In some embodiments, the system may determine a variability of the waveform. Variability is a term in the art that may be operative to provide an indication of a cognitive skill level. For example, excessive variability may be indicative of cognitive decline. In a further embodiment, variability may be operative to inform at least one aspect of a training. The aforementioned uses are provided for example only and any use of variability may be utilized.

Variability may be related to, for example, and without limitation, may be determined from the frequency or the amplitude dimension of the waveform. However, a person of ordinary skill in the art will recognize that variability may be utilized to express properties related to any aspect of the waveform through any methods as needed or desired. In some embodiments, the variability of the waveform may be compared to the variability from other iterations of motivational interviews. Comparing changes in the variability of the waveform over time may be operative to track changes in the user's cognitive skills. In some embodiments, the changes in the variability may be operative to detect a change in the user's overall wellbeing. For example, and without limitation, the changes may relate to an illness, a change in the user's life, or any other area that a person of ordinary skill in the art may recognize.

In one embodiment, the variability may be related to the cognitive profile of the user. In another embodiment, the variability may be related to the user's well-being phenotype. In yet a further embodiment, the variability may be related to more than one area of the user's assessment profile.

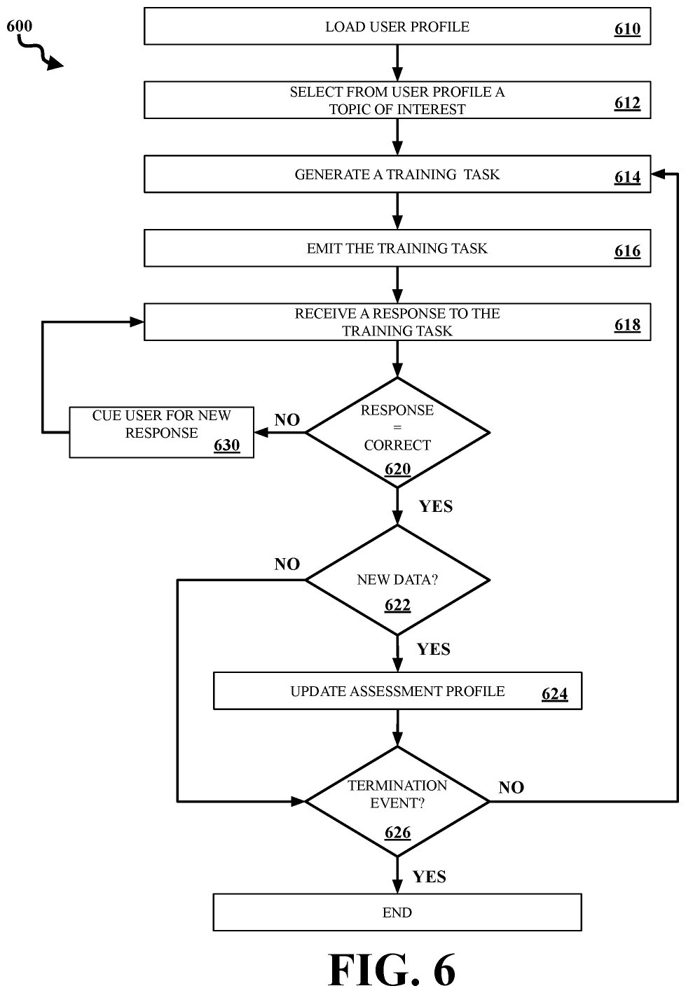

In some embodiments, the system may utilize the changes in variability in a personalized training illustrated in FIG. 6, below. For example, the system may increase/decrease the frequency, duration, or difficulty of the personalized training occurring to the changes in variability.

The step of transforming the waveform to the frequency-domain signal 520 may convert the waveform from time-based to frequency-based. The frequency-domain signal may represent how much of the waveform lies within each of a range of frequencies. This transformation may be done using any method known in the art operative to transform signals. For example, the waveform may be converted using Fourier transformation, Laplace transformation, or any other method known in the art.

Following the step of transforming the waveform, the frequency-domain signal 520, the spectrum may be analyzed 522. In particular, the spectrum of frequencies in the frequency-domain signal may be analyzed.

The frequency-domain signal may comprise a power spectral density and at least one frequency band. Power spectral density is a term in the art that describes frequencies over a continuous range, such as a frequency band. The at least one frequency band may, for example, be a limited frequency range. For example, the at least one frequency band may comprise the limited frequency range of any of 20 to 60 Hz, 60 to 250 Hz, 250 Hz to 500 Hz, 500 to 2,000 Hz, 2,000 Hz to 4,000 Hz, 4,000 to 6,000 Hz, or 6,000 Hz to 20,000 Hz. A person of ordinary skill will recognize that the aforementioned frequency ranges correspond with known frequency bands sub bass, bass, low mids, midrange, upper mids, presence, and highs respectively. However, any other limited frequency ranges, whether as needed or desired may be used to practice the invention.

The frequency domain signal may further comprise at least one harmonic, as understood by one of ordinary skill in the art. The system may be operative to determine a phase relationship of the at least one harmonic. A person of ordinary skill in the art will appreciate that the at least one harmonic and the phase may be representative of the amplitude and frequency of the response received to the motivational interview.

A person of ordinary skill in the art will appreciate that the frequency-domain signal may be utilized to identify individual words. In one embodiment, the frequency-domain signal may be a spectrogram. The spectrogram is known in the art as one form of audio processing used to identify words and phrases.

While steps 514, 516, 518, 520, and 522 are described consecutively, this is for example only and may be done in any order or even simultaneously.

The step of updating the user's assessment profile may comprise updating any of the emotional profile, cognitive profile, topics of interest, sentiment orientation, and well-being phenotype. As previously discussed, the emotional state, cognitive skills, and sentiment orientation of the user may be determined using the methods described in FIGS. 3-5. It is contemplated that the well-being phenotype may also be determined using the methods described in FIGS. 3-5.

In some embodiments, the well-being phenotype may comprise information relating to any of the user's physical well-being, cognitive well-being, psychological well-being, social well-being, or any other information as needed or desired. As such, the well-being phenotype may comprise information related to any of the assessment profiles. For example, and without limitation, the well-being phenotype may comprise any of the emotional profile, the cognitive profile, the interest profile, and the sentiment orientation. Thus, the well-being phenotype may comprise results from any of the analysis conducted in step 312 of FIG. 3. Including, without limitation the emotional state and sentiment orientation described in FIGS. 4 and 5. Further, the responses to the at least one question in the motivational interview may be further utilized to represent the well-being phenotype. It is further contemplated that the well-being phenotype may comprise data extracted from the personalized training, discussed in more detail below with reference to FIG. 6.

Continuing with the well-being phenotype, it may be represented in any manner as needed or desired. In some embodiments, the well-being phenotype may be represented through a scaled numerical representation. In another embodiment, the well-being phenotype may be abstractly represented through words, phrases, colors, emoticons, or any other form as desired.

In one embodiment, the well-being phenotype may be presented graphically. It is contemplated that graphical representation may permit a clear comparison of changes to the well-being phenotype over time. For example, and without limitation, the well-being phenotype may be a multi-axis graph wherein each axis represents a different area. Such as and without limitation to, a four-axis graph wherein each axis relates to the user's physical well-being, cognitive well-being, psychological well-being, and social well-being. A person of ordinary skill in the art will appreciate that the well-being phenotype may be displayed in a wide variety of manners and the aforementioned examples are provided as non-limiting examples.

The emotional profile may comprise any of the emotional states determined in FIG. 3-5. Further, the emotional profile may comprise emotional states determined by other iterations of motivational interviews.

In one exemplary embodiment, the system may create a graphical representation of the emotional state of the user. The graphical representation may be any graphical representation known in the art, including, for example, a graph, a table, a chart, a plot, a map, or any other graphical representation as needed or desired. In one exemplary embodiment, the graphical representation may be a multi-dimensional model. For the sake of brevity, comparing the at least one pre-selected emotion to another is discussed using three pre-selected emotions, but may be practiced with any number of predetermined emotions. In one embodiment, the three pre-selected emotions may be presented as a three-dimensional space model. In a further embodiment, the three pre-selected emotions may be presented as a heat map. The heat map may combine the three pre-selected emotions, each of the emotions comprising a positive and negative dimension.

In another exemplary embodiment, the graphical representation may be a polar plot. It is contemplated that the polar plot may be advantageous to display a plurality of the at least one predetermined emotion at once. The polar plot may list complementary emotions, such as happy and content or sad and disgust, near one another. The polar plot may further list contradicting emotions, such as happy and sad or bored and excited on opposing points along the polar plot. The emotional state may be displayed as a continuous line on the polar plot connecting the weight of the at least one emotion together. The aforementioned embodiment is well understood by a person of ordinary skill in the art and is thus only briefly discussed for the sake of brevity.

The cognitive profile may comprise information relating to the user's at least one cognitive skill. In particular, the cognitive profile may comprise the skill level of each of the at least one cognitive skill. In some embodiments, the skill level may be represented using a graphical representation. For example, and without limitation, the cognitive skill may be illustrated using a bar chart, a bell curve, a color chart, or any other graphical representation known in the art. In another embodiment, the skill level of each of the at least one cognitive skill may be represented using in any manner as needed or desired. In another embodiment, the at least one cognitive skill may be represented by a percentage, letter, emoticon, color, or any other means able to represent a skill level.

As previously discussed, with reference to FIG. 4, the system may determine the sentiment orientation of the user. In one embodiment, the system may be operative to generate a semantic word map operative to represent the sentiment orientation of the user. The semantic word map may display at least one word and/or phrase related to the sentiment orientation. In one embodiment, the system may be operative to identify connections between the words extracted during the linguistic analysis 400 (FIG. 4). In some embodiments, these connections may be utilized to establish a vocabulary of the user. The vocabulary of the user may comprise typical uses of words and/or phrases received from the user and their connotation. In such an embodiment, the system may be operative to identify meaning-based connections between words and/or phrases in the vocabulary of the user.

In one embodiment, the system may utilize automatic text summarization to extract words and/or phrases relevant to the user's assessment profile. In some embodiments, the extracted words and/or phrases may be based on a frequency of the words and/or phrases. In a further embodiment, the words and/or phrases may be extracted depending on other words and/or phrases that appear in conjunction.

The extracted words and/or phrases may be operative to update any assessment profile. In some embodiments, the assessment profile may comprise at least one word cloud. A person of ordinary will recognize that word clouds are well known in the art and any form of word cloud as needed or desired may be utilized. In one embodiment, the at least one word cloud may be related to the extracted words and/or phrases. In some embodiments, any words of the at least one word cloud may comprise a synonym to the extracted words and/or phrases. In one embodiment, the synonym may be any of the words and/or phrases in the vocabulary of the user. In another embodiment, the words and/or phrases in the vocabulary of the user may determine the synonym.

The aforementioned graphical representations may be displayed on the electronic device 110 discussed in FIG. 2. However, in another embodiment, the graphical representation may be transmitted to the user's account. It is contemplated that the user account may be accessed using any electronic device operative to access the user account. Further, the graphical representation may be transmitted to the email or physical address associated with the user account.

In yet a further embodiment, the graphical representation may be stored at the server 120 shown in FIG. 1. The graphical representation may then be displayed upon a request from any device to access the graphical representation from the server 120.

While the aforementioned embodiments utilize graphical representations for representing aspects of the assessment profile, this may be represented numerically or even in the abstract.

In one embodiment, the interest profile may comprise a list of the at least one topic of interest determined by the motivational interview. In some embodiments, the list of the at least one topic of interest may comprise an order. The order may be any order as needed or desired, including without limitation, a level of passion relating to the at least one topic of interest, alphabetically, or sequentially. It is contemplated that interest profile may, in some embodiments, comprise a graphical representation in any form as needed or desired.

A person of ordinary skill in the art will appreciate that any area of the assessment profile may be related to another area of the assessment profile. As such, while these areas are discussed independently, they may inform another area of the assessment profile.

While conducting the analysis 312 of the response to the at least one question is described separately from the motivational interview, it is contemplated that the motivational interview may comprise the analysis (step 312). In some embodiments, the system may conduct the personalized training to train at least one cognitive skill. In an exemplary embodiment, the at least one cognitive skill may be a conceptual cognitive skill. However, the system may be operative to train any cognitive skill as needed or desired.

One exemplary method for conducting personalized training is shown in FIG. 6. The personalized training comprises the steps of loading a user profile 610, selecting at least one topic of interest from the user profile 612, generating a training task 614, presenting the training task 616 to the user, and receiving a response to the training task 618.

The user profile may be the user profile described in FIG. 3. Thus, it is contemplated that the at least one topic of interest may be any of the topics of interest in the interest profile. In one embodiment, the at least one topic of interest may be determined according to the linguistic analysis 400 described in FIG. 4. However, in some embodiments, the at least one topic of interest may be determined by the paralinguistic analysis 500 described in FIG. 5. In another embodiment, the at least one topic of interest may be specified by the user. In a further embodiment, the at least one topic of interest may be specified by the user according to a predetermined list of topics of interest. In yet another embodiment, the at least one topic of interest may be determined according to the assessment profile associated with the user account. This information may, without limitation, be any of the user's topics of interest, content connections, cognitive profile, emotional profile, and well-being phenotype.

The training task generated by the system (step 614) may relate to the at least one topic of interest. In some embodiments the at least one topic of interest may have at least one associated training task. In some embodiments, the at least one associated training task may be a list of associated training tasks. In some such embodiments, the system may be operative to take each of the associated training tasks from the list in turn. In another embodiment, the associated training task may be taken in any order from the list. In yet a further embodiment, the response to the at least one training task may be operative to determine another training.

It is contemplated that generating the training task specific to the user's interests may be operative to increase efficacy of the system. Generating training tasks related to the at least one topic of interest may be operative to capture the user's interest, thus providing optimal cognitive stimulation.

The training task may be presented to the user in any manner operative to engage the user as needed or desired. In one embodiment, the training task may be audibly presented through the speaker of the exemplary electronic device described in FIG. 2. In another embodiment, the training task may be displayed on the graphical user interface of the electronic device. In yet a further embodiment, the training task may comprise audible and visual components and thus may be presented using a combination of interfaces.

The training task may be any prompt operative to train the user. In one embodiment, the training task may comprise a training prompt. The training prompt may comprise information necessary to answer the question. In another embodiment, the training task may comprise a question. In some embodiments, the question may be based on the user's pre-existing knowledge. For example, the training prompt may be an arithmetic problem and the question may instruct the user to answer the question. In another embodiment, the question may be based on the training task. Thus, the answer to the question may be presented alongside the question. For example, the training task may comprise a sequence of letters or numbers and the question may correspond to the letters in the sequence. In yet another embodiment, the training prompt may comprise an instruction operative to communicate a desired response from the user.

In another embodiment, the training prompt may be a sound, such as a beep or a melody For example, when the training prompt is the sound, the instruction may comprise instructions on the desired response, such as a word, phrase, or even a lack of affirmative response, to the training prompt. A person of ordinary skill in the art will appreciate that the aforementioned embodiments are provided as examples only and that any training prompt as needed or desired may be utilized.

Following the step of presenting the training task 616, the response to the training task may be received 618. It is contemplated that the response may be received at any electronic device operative to perform the method. In the exemplary embodiment of the electronic device in FIG. 2, the response may be received at the audio interface 206. In some embodiments, the response may be a word or phrase, however, in other embodiments, the response may be any affirmative response. In yet a further embodiment, the response may be a lack of the affirmative response, for example, silence.

The response received may be received in any form that may be received. In some embodiments, the system may be operative to receive an audio response. The audio response may, without limitation, be speech, a percussive sound, or any other sound. For example, the system may receive the audio response at the microphone described on the exemplary electronic device in FIG. 2. In one embodiment the response may be a physical response, such as a selection made on the graphical user interface. In another embodiment, the response may be a combination of physical and audio responses. Any response known in the art that may be received may be used and the aforementioned embodiments are provided as non-limiting examples only.

The training task may comprise a correct response specific to each training task. In one embodiment, the correct response may be identifying a change in the training task, for example, a change in the melody. Further exemplary examples of training tasks are provided as working examples below. However, a person of ordinary skill will recognize that the examples are provided as non-limiting examples and the correct response may be any correct response as needed or desired.

The system may be operative to determine if the response to the training task is the correct response 620. In one embodiment, wherein the response to the training task is not the correct response, the system may cue the user for a new response 630. In some such embodiments, the system may repeat the training task to the user. However, in another such embodiment, the system may inform the user the response to the training task is incorrect and prompt the user for the new response. In yet a further embodiment, the system may rephrase the training task. In yet another embodiment, the system may present a hint to the training task. The aforementioned embodiments are provided for example only and any cues as needed or desired may be utilized.

The new response to the training task may be received 618 and the system may determine if the new response is the correct response 620. If the new response is not the correct response, the system may continue to cue the user for the new response 630. However, in some embodiments, not illustrated, the system may generate a new training prompt. In yet another embodiment not shown, the system may terminate the personalized training when the response to the training task is incorrect. It is contemplated that terminating the personalized training may occur after a predetermined number of incorrect responses to the training prompt. The predetermined number of incorrect responses may be any number, including for example, one, two, three, four, or even more. In some embodiments, the predetermined number may be specific to each training task. In another embodiment, the predetermined number may be specific to the user.

In some embodiments, the response to the training task may be partially correct. In some such embodiments, the system may task the user for the new response. However, in other such embodiments, the system may treat the response to the training task as the correct response.

Continuing with the embodiment illustrated in FIG. 6, when the response to the training task is correct, the system may determine if new data is present in the response to the training prompt 622. The new data may be any data related to the assessment profile. For example, the new data may be any of the user's emotional state, cognitive skill, topics of interest, sentiment orientation, or well-being.

The new data may be extracted from the response to the training task in any manner. In one embodiment, the new data may be extracted according to the linguistic analysis 400 displayed in FIG. 4. In another embodiment, the new data may be extracted according to the paralinguistic analysis 500 displayed in FIG. 5. However, the aforementioned embodiments are provided as examples only and any manner of extracting data may be utilized.

When the response to the training task comprises new data, the system may update the user's assessment profile (step 624). More particularly, the system may store the new data in the user's assessment profile. The new data may be related to any of the emotional profile, cognitive profile, interest profile, semantic map, and well-being phenotype. It is contemplated that the system may update any of the assessment profile areas that the new data may be related to. For example, and without limitation when the new data comprises emotional states, the emotional profile may be updated.

It is contemplated that updating the user's assessment profile 624 may utilize any of the aforementioned discussed areas of the user's assessment profile. However, for the sake of brevity each embodiment is not repeated here and reference is made to the previously disclosed embodiments.

However, it is contemplated that not all responses to the training task may comprise new data. In such instances, the system may determine if a termination event has occurred 626. Additionally, the system may determine if the termination event has occurred 626 following the step of updating the user's assessment profile 624.

The termination event may be any event that signals an end of the personalized training 600. In one embodiment, the termination event may be a negative emotional state. The negative emotional state may, for example, and without limitation, be informed by the new data from the previous steps (steps 622 and 624). It is contemplated that negative emotional states may, as non-limiting examples only, comprise any of frustration, anger, embarrassment, melancholy, or any other negative emotion. In another embodiment, the termination event may be any of a passage of time, completion of a predetermined series of questions, a request to terminate, or any other event that may be contemplated.

It is contemplated that multiple termination events may be present and upon the occurrence of any of the termination events the system may end the personalized interview.

If no termination event has occurred, the system may generate a new training task 614 and repeat the aforementioned steps. This may continue until the termination event has occurred.

In order to more fully understand the personalized training performed by the system and method disclosed herein, exemplary working examples are presented. It should be noted that these working examples are non-limiting examples of personalized training that may be implemented.

### Working Example #1

Working example #1 relates to the personalized training as illustrated in FIG. 6. In this working example, the personalized training may be configured to target the user's cognitive skill of reasoning. More particularly, the training task may target the user's ability to adapt behaviors and thought to new, changing, or unexpected events, referred to generally as the cognitive skill of shifting.

In this working example, the training prompt may be a series of beeps emitted through the at least one speaker of the electronic device. The series of beeps may comprise at least two distinct types of beeps, such as beeps of different frequencies and/or durations. For the interest of brevity and clarity, this working example will be discussed with reference to only two distinct types of beeps, referred to as beep A and beep B. However, a person of ordinary skill in the art will appreciate that the at least two distinct types of beeps may be any number of beeps.

The training task may further comprise an instruction on how to respond to the series of beeps. For example, the instruction may instruct the user to clap, tap, or make a similar noise when beep A is emitted and do nothing when beep B is emitted. In another example, the user may be instructed to clap, tap, or make a similar noise when beep B is emitted and do nothing when beep A is emitted. The correct answer will be recognized as the user's response or lack thereof when the various beeps are emitted.

The series of beeps may comprise any number of beeps A and B and may arrange the beeps in any order as needed or desired. In one example, the series of beeps may comprise A, A, B, A, B, B, B, A, B, and B. However, a person will appreciate that this is only one of innumerable combinations and that any combination may be used to carry out the invention.

A delay may occur between each beep emitted to permit the system to receive the user's response to the beeps. The delay may be any length of time and may, in some embodiments, be specific to the user. For example, the delay may shorten after the system receives consecutive correct responses and lengthen the delay after the system receives consecutive incorrect responses. It is contemplated that this may gradually increase, or even decrease, the difficulty of the training task. In another embodiment, the delay may remain constant throughout the entirety of one iteration of personalized training and the duration of the delay may be varied between iterations of personalized training.

### Working Example #2

Working example #2 relates to one training task that may be presented as part of the personalized training illustrated in FIG. 6. In this working example, the training task may be configured to train the user's auditory short-term memory.

The training prompt may comprise a melody fragment and at least one melody. One of the at least one melodies may comprise the melody fragment. In some embodiments, the at least one melody comprising the melody fragment may be the melody fragment in its entirety. However, in another embodiment, the melody fragment may be less than all of the at least one melody. The system may emit an instruction to identify which of the at least one melodies comprise the melody fragment. The system may then emit one of the at least one melody and receive a response to the emitted at least one melody. The response may, for example, identify whether the at least one melody comprises the melody fragment. The response when the at least one melody does not comprise the melody fragment may be the absence of the response or even a negative response.

In some embodiments of this working example, the system may further instruct the user to identify where the melody fragment is located in the at least one melody. For example, the melody fragment may be located at a beginning, middle, or end of the at least one melody and the correct response may correlate to the location of the melody fragment.

### Working Example #3

Working example #3 relates to one embodiment of the training tasks that may be presented as part of the personalized training discussed with reference to FIG. 6. In this working example, the cognitive skill targeted by the training task may be short-term memory.

The training prompt may comprise a series of numbers, letters, colors, or even names. In the interest of brevity, the training task is discussed with reference to the series of numbers, however, any series may be utilized.

The system may emit instructions to the user that request the user to repeat the series of numbers. In one example, and without limitation the training prompt may be “Hey, can you repeat this after me? 7-6-0-8-3-1. Your turn!” It is contemplated that the correct answer may be the repetition of the sequence of numbers.

The system may be operative to adjust the difficulty of the provided training task. For example, the series of numbers may be longer to increase the difficulty of the provided training task and shorter to decrease the difficulty of the provided training task. In another example, the speed at which the numbers are emitted may be varied. In yet another example, the complexity of the numbers may be increased.

### Working Example #4

In working example #4, the training task may be configured to target the cognitive skill of working memory. The training prompt may comprise a sequence of stimuli that are presented to the user. The stimuli may be letters, numbers, words, phrases, or any other stimuli as needed or desired. For brevity, the example stimuli are referred to as letters and is not limiting. For example, the sequence of letters may be “T-L-D-V-B-O-P-Q-L-C-K-Q-T-A-R-K.”

Next, the system may emit a question to the user to identify one stimulus in the sequence. For example, the system may ask the user to identify the stimulus in the fifth position in the sequence and the correct may be letter “B.” In another example, the system may ask the user to identify where a designated stimulus is in the sequence, such as the position of letter C and the correct answer may be “8.” In another example, the system may ask the user to identify one stimulus in relation to another stimulus, such as “what is the letter following letter V?” wherein the correct letter is the answer “B.” Of course any prompt that targets working memory may be utilized and the aforementioned examples are provided without limitation. It is contemplated that multiple prompts may be provided for the same sequence of letters.

The system may be operative to adjust the difficult of the working example #4. For example, the length of the sequence of letters may be increased or decreased according to the user's cognitive skill level.

### Working Example #5

In this working example, the training task from the personalized training in FIG. 6 may be configured to train the user's memory. More particularly, the training task may be a computation span task.

This training prompt may comprise a series of arithmetic problems. A first arithmetic problem in the series of arithmetic problems may be presented through the at least one speaker. The system may emit an instruction to the user to solve the first arithmetic problem and recall the answer to the first arithmetic problem. Next, the system may emit each of the arithmetic problem in the series of problems and an instruction to solve each of the arithmetic problems in the series of problem and to recall the answer to each of the arithmetic problems.

In some embodiments of this working example, the system may emit a request that the user repeat back the answer to each arithmetic problem in the series that have been presented to the user. The system may emit the request after any number of the arithmetic problems in the series. In some embodiments, the request may be issued after each of the arithmetic problems in the series. In other embodiments, the request may be issued after every two, three, four, six, or even other numbers. Of course, the request may be any request, such as requesting the user to repeat the answer to an arithmetic problem in a random position, further arithmetic problems that utilize the answers to previous arithmetic problems, or any other request as needed or desired.

In one example of this working problem, the first arithmetic problem in the series may be “1+8” wherein the correct answer is 9. Next, the system may emit a second arithmetic problem, such as “7-2,” wherein the correct answer is 5. The system may emit the request asking the user to provide the answers to the series of arithmetic problems in order. As such, the system may receive the user's response and may determine if it is the correct response of “9-5.”

Indeed, the system may be operative to adjust the difficulty, by increasing or decreasing the frequency of the request, of the training task according to the skill level of the user.

Turning to FIG. 7, an exemplary embodiment of a feedback loop 700 carried out by the system to assess and train cognitive abilities is shown. This feedback loop 700 shows the user profile 232 may inform the motivational interview 234 and a cognitive profile 710. The motivational interview 234 may inform the paralinguistic analysis 500 and the linguistic analysis 400. The paralinguistic analysis 500 and the linguistic analysis 400 may inform the cognitive profile 710 and an emotional profile 712. The linguistic analysis 400 may further inform the interest profile 716. The cognitive profile 710 and the emotional profile 712 may then inform a user phenotype 714 and the user phenotype 714 may then inform the personalized training 600. The personalized training 600 may be further informed by the interest profile 716. The personalized training 600 may then inform a well-being phenotype 718. The user profile 232 may be further informed by the personalized training 600.

Of course, the embodiment shown in FIG. 7 is only one feedback loop that may be performed by the system.

### CONCLUSIONS, RAMIFICATIONS, AND SCOPE

While certain embodiments of the invention have been illustrated and described, various modifications are contemplated and can be made without departing from the spirit and scope of the invention. For example, the system may be utilized as a testing means for visually impaired or illiterate individuals. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the customizable cognitive rehabilitation method and system with which that terminology is associated. In general, the terms used in the following claims should not be constructed to limit the customizable cognitive rehabilitation method and system to the specific embodiments disclosed in the specification unless the above description section explicitly defines such terms. Accordingly, the actual scope encompasses not only the disclosed embodiments, but also all equivalent ways of practicing or implementing the disclosed system, method and apparatus. The above description of embodiments of the customizable cognitive rehabilitation method and system is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the customizable cognitive rehabilitation method and system.

What is claimed is:

1. A computer implemented method for conversational cognitive stimulation comprising:
by a computer system comprising at least one speaker, at least one microphone, and a processor:
loading a user profile comprising a user's identifying information and an assessment profile;
conducting a motivational interview, comprising the steps of:
loading at least one question;
emitting through the at least one speaker the at least one question;
receiving on the at least one microphone a response to the at least one question;
conducting an analysis of the motivational interview, comprising the steps of:
conducting a linguistic analysis of the motivational interview;
conducting a paralinguistic analysis of the motivational interview; and
updating the user's assessment profile.

2. The method of claim 1, wherein the assessment profile comprises an emotional profile, a cognitive profile, an interest profile, a sentiment orientation, and a well-being phenotype.

3. The method of claim 1, wherein conducting the linguistic analysis of the motivational interview comprises the steps of:
converting the response to the at least one question received on the at least one microphone to text;
tagging distinct words in the text;
converting each word tagged to its root form;
determining an emotional state of the user; and
comparing the at least one emotion to an emotion analysis database.

4. The method of claim 1, wherein conducting the linguistic analysis of the motivational interview comprises the steps of:
converting the response to the at least one question to text;
tagging distinct words in the text;
converting each word to its root form;
determining a sentiment of the text; and
determining the sentiment orientation of the user.

5. The method of claim 1, wherein conducting the paralinguistic analysis comprises the steps of:
transforming the response to the at least one question to a waveform;
analyzing a time dimension of the waveform;
analyzing a frequency of the waveform;
analyzing an amplitude dimension;
transforming the waveform to a frequency-domain signal;
analyzing a spectrum of the frequency-domain signal; and
determining an emotional state of the user.

6. The method of claim 1, wherein the step of conducting the analysis of the motivational interview is operative to identify at least one topic of interest of the user.

7. The method of claim 6, further comprising conducting a personalized training comprising the steps of:
selecting one of the at least one topic of interest;
generating at least one training task related to the selected at least one topic of interest;
emitting through the at least one speaker the at least one training task to the user;
receiving a response to the training task;
responsive to the response being correct, determining if the response comprises new data;
updating the user's assessment profile with the new data; and
repeating the steps until a termination event occurs.

8. The method of claim 7, wherein the termination event is selected from a group consisting of detecting a negative emotional state, a passage of time, completion of a predetermined series of questions, and a request to terminate.

9. The method of claim 7, wherein the personalized training is operative to target at least one cognitive skill selected from a group comprising attention, perception, memory, reasoning, coordination, and language.

10. A system for conversational cognitive stimulation comprising:
a first electronic device comprising at least one speaker, at least one microphone, and a processor, operative to:
load a user profile on the first electronic device, the user profile comprising a user's identifying information and assessment profile;
conduct a motivational interview, comprising the steps of:
loading at least one question;
emitting the at least one question through the at least one speaker;
receiving, through the at least one microphone, a response to the at least one question;
conduct an analysis of the motivational interview; and
update the user's assessment profile.

11. The system of claim 10, wherein the assessment profile comprises an emotional profile, a cognitive profile, an interest profile, a sentiment orientation, and a well-being phenotype.

12. The system of claim 10, wherein the step of conducting the analysis comprises a linguistic analysis comprising the steps of:
converting the response to the at least one question to text;
tagging distinct words in the text;
converting each word tagged to its root form;
determining an emotional state of the user;
comparing the at least one emotion to an emotion analysis database;
determining a sentiment of the text; and
determining the sentiment orientation of the user.

13. The system of claim 10, wherein the step of conducting the analysis of the motivational interview comprises a paralinguistic analysis comprising the steps of:
transforming the response to the at least one question to a waveform;
analyzing a time dimension of the waveform selected from a group consisting of analyzing a duration of utterances, articulation rate, and a number and duration of pauses;
analyzing a frequency of the waveform wherein analyzing the frequency of the waveform comprises a pitch;
analyzing an amplitude dimension comprising wherein the step of analyzing the amplitude dimension is selected from a group consisting of analyzing a power of the waveform, a vocal shimmer, jitter, pitch perturbation, vocal entropy, quality, intensity, and loudness;
transforming the waveform to a frequency-domain signal;
analyzing a spectrum of frequencies in the frequency-domain signal, the spectrum of frequencies may comprise a power spectral density and at least one frequency band; and
determining an emotional state of the user.

14. The system of claim 10, wherein the step of conducting the analysis of the motivational interview is operative to determine at least one topic of interest of the user.

15. The system of claim 14, wherein the processor is further operative to conduct a personalized training, comprising the steps of:
selecting one of the at least one topic of interest;
generating at least one training task related to the selected at least one topic of interest;
presenting the at least one training task to the user through the at least one speaker;
receiving at least at least one microphone, a response to the training task;
responsive to the response being correct, determining if the response comprises new data;
updating the user's assessment profile with the new data; and
repeating the steps until a termination event occurs.

16. The system of claim 15, wherein the termination event is selected from a group consisting of detecting a negative emotional state, a passage of time, completion of a predetermined series of questions, and a request to terminate.

17. The system of claim 13, wherein the personalized training is operative to target at least one cognitive skill selected from a group consisting of attention, perception, memory, reasoning, coordination, and language.

18. The system of claim 17, wherein the step of updating the user's assessment profile comprises updating a cognitive profile with the at least one cognitive skill.

19. A non-transitory, tangible computer-readable medium having stored thereon computer-executable instructions, which, when executed by a computer processor, enable performance of the method comprising:
loading, via a first computing device, a user profile comprising a user's identifying information and assessment profile comprising an emotional profile, a cognitive profile, an interest profile, a sentiment orientation, and a well-being phenotype;
conducting a motivational interview, comprising the steps of:
loading at least one question;
presenting the at least one question;
receiving a response to the at least one question;
conducting a linguistic analysis of the motivational interview;
conducting a paralinguistic analysis of the motivational interview;
determining from the linguistic and paralinguistic analysis at least one topic of interest; and
in response to the linguistic and paralinguistic analysis, updating the emotional profile with the emotional state, updating the interest profile with the at least one topic of interest, and updating the sentiment orientation with the sentiment of the text.
